(19) United States
(12) Patent Application Publication
(10) Pub. No.: US 2021/0038363 A1
(43) Pub. Date: Feb. 11, 2021

(54) MEDICAL SELF-EXPANDABLE STENT

(71) Applicants: THE ASAN FOUNDATION, Seoul (KR); UNIVERSITY OF ULSAN FOUNDATION FOR INDUSTRY COOPERATION, Ulsan (KR)

(72) Inventor: Do Hyun PARK, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 17/080,979

(22) Filed: Oct. 27, 2020

Related U.S. Application Data
(63) Continuation of application No. PCT/KR2020/008846, filed on Jul. 7, 2020.

(30) Foreign Application Priority Data
Jul. 8, 2019 (KR) 10-2019-0082258

(51) Int. Cl. A61F 2/04; A61F 2/82; A61B 18/18

(52) U.S. Cl. CPC A61F 2/04; A61F 2/82; A61B 2018/1807; A61F 2002/045; A61F 2210/0057; A61B 18/18

(57) ABSTRACT

Provided is a medical self-expandable stent which includes a stent body that is self-stretched or expanded by heat and emits the heat, a stretchable light emitting diode (LED) that is stacked to be longitudinally spread on the stent body and emits the heat by converting electric energy into optical energy, and a thin-film formation part that includes a thin film to fix the stretchable LED to the stent body, and is self-expanded by the heat.

### CROSS-REFERENCE TO RELATED APPLICATIONS

The present application is a continuation of International Patent Application No. PCT/KR2020/008846, filed on Jul. 7, 2020, which is based upon and claims the benefit of priority to Korean Patent Application No. 10-2019-0082258 filed on Jul. 8, 2019. The disclosures of the above-listed applications are hereby incorporated by reference herein in their entirety.

### BACKGROUND

Embodiments of the inventive concept described herein relate to a medical self-expandable stent, and more particularly, relate to a medical self-expandable stent capable of regenerating and treating a target part by mounting a stretchable LED, inserting a stent body, which is self-expanded, into the target part of a living tissue, and irradiating optical energy of the stretchable LED.

There are 600 million obese patients worldwide, which include domestic patients, and 36.5% of the US population is overweight or obese, and the prevalence rate is increasing every year.

In addition, diabetes patients are reported to about 380 million people in 2014, and type 2 diabetes patients are reported to be about 90% of them.

Most of obese patients have diabetes. In this case, effective obesity treatment is difficult with only obesity drug treatment and exercise therapy. In addition, drug-related side effects are significantly produced, and there are many drug abuses.

Recently, there has been introduced a procedure for a duodenal mucosal regeneration to cure diabetes by regenerating a normal cell after destroying diabetes-related cells present in part 2 and part 3 of the duodenum through high-frequency treatment.

However, in the case of such a duodenal mucosal regeneration through the high-frequency treatment, since water is filled in a target part of the duodenum, the high-frequency is irradiated, and the mucosal cell at the target part is destroyed, not only may the mucosal cell at the target part be excessively damaged by the higher-temperature heat, but duodenal stenosis may be caused.

### SUMMARY

Embodiments of the inventive concept provide a medical self-expandable stent capable of performing treatment by ablating a mucosal cell at a target part of a living tissue using optical energy of a stretchable LED to regenerate a mucosal cell of a duodenum without the damage to a duodenum tissue, and of reducing the complications resulting from the treatment by preventing duodenal stenosis.

Embodiments of the inventive concept provide a medical self-expandable stent capable of consecutively treating a target part of a living tissue by wirelessly transmitting power to a stent inserted into a human body.

According to an exemplary embodiment, a medical self-expandable stent may include a stent body that is self-stretched or expanded by heat and emits the heat, a stretchable light emitting diode (LED) that is stacked to be longitudinally spread on the stent body and emits the heat by converting electric energy into optical energy, and a thin-film formation part that includes a thin film to fix the stretchable LED to the stent body and is self-expanded by the heat.

In this case, the medical self-expandable stent may further include a stretchable battery that is provided to be surrounded by the thin-film formation part and longitudinally spread on the stent body, and supplies electric energy to the stretchable LED.

The medical self-expandable stent may further include a communication unit that is stacked on the stent body and receives a control command from an outside through wireless communication, and a controller that is stacked on the stent body and turns on or turns off the stretchable LED in response to the control command received from the communication unit.

The medical self-expandable stent may include an auxiliary thin-film formation part that includes a thin film between the stretchable LED and the stent body, and is self-expanded by the heat.

The medical self-expandable stent may include a radio frequency (RF) signal generator that generates an RF signal, an RF amplifier that amplifies the RF signal, which is output from the RF signal generator, to specific power, a power transmitter that wirelessly transmits the RF signal amplified to the specific power, and a power receiver that converts the RF signal received from the power transmitter into power and supplies the power to the stretchable battery.

The medical self-expandable stent may further include a circuit board on which the stretchable LED is mounted, and a radiating plate that is provided on the circuit board, and radiates the heat transferred from the stretchable LED to the circuit board.

A plurality of stretchable LEDs may be provided, and may emit light having mutually different wavelengths.

The stretchable battery may include a thin-film pouch cell or a flexible battery.

The thin-film formation part may be formed by performing electrospraying or electrospining for silicone.

The thin-film formation part may further include magnesium (Mg).

The auxiliary thin-film formation part may be formed by performing electrospraying or electrospining for silicone.

The auxiliary thin-film formation part may further include magnesium (Mg).

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a medical self-expandable stent, according to an embodiment of the inventive concept;

FIG. 2 is a sectional view of FIG. 1;

FIG. 3 is a block diagram for the control of a medical self-expandable stent, according to an embodiment of the inventive concept;

FIG. 4 is a perspective view illustrating a medical self-expandable stent, according to another embodiment of the inventive concept;

FIG. 5 is a schematic view illustrating a medical self-expandable stent inserted into part 2 of the duodenum, according to an embodiment of the inventive concept; and

FIGS. 6 to 10 are views illustrating the procedure of inserting a medical self-expandable stent into part 2 of the duodenum, according to an embodiment of the inventive concept.

### DETAILED DESCRIPTION

Advantage points and features of the inventive concept and a method of accomplishing thereof will become apparent from the following description with reference to the following drawings, wherein embodiments will be described in detail with reference to the accompanying drawings. However, the inventive concept may be embodied in various different forms, and should not be construed as being limited only to the illustrated embodiments. Rather, these embodiments are provided as examples so that the inventive concept will be thorough and complete, and will allow those skilled in the art to fully understand the scope of the inventive concept. The inventive concept may be defined by scope of the claims.

The terminology used herein is provided for explaining embodiments, but the inventive concept is not limited thereto. Herein, singular terms are intended to include plural forms as well, unless the context clearly indicates otherwise. Furthermore, it will be further understood that the terms “comprises”, “comprising,” “includes” and/or “including”, when used herein, specify the presence of stated components, but do not preclude the presence or addition of one or more other components. The same reference numerals will be assigned to the same component throughout the whole specification, and “and/or” refers to that components described include not only individual components, but at least one combination of the components.

Unless otherwise defined, all terms (including technical and scientific terms) used herein have the same meaning as commonly understood by those skilled in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, the inventive concept will be described in detail with reference to accompanying drawings.

Before the description, although the present embodiment will be described in that a medical self-expandable stent according to the inventive concept is inserted into part 2 of the duodenum, the medical self-expandable stent according to the inventive concept may be applied to a human lumen and/or a human cavity, for example, the gastrointestinal tract, esophagus, stomach, pylorus, lungs, bladder, nasopharynx, colon, airway, or oral cavity.

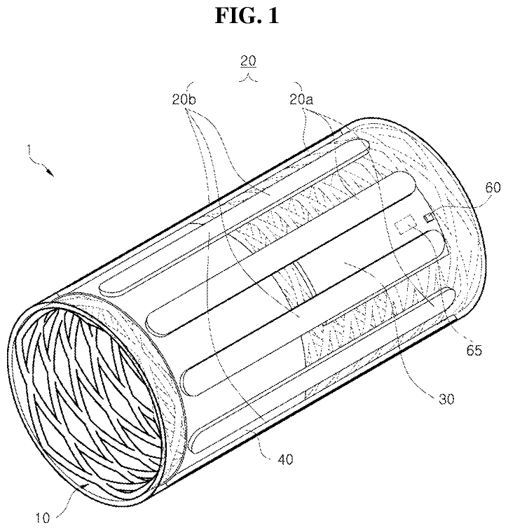

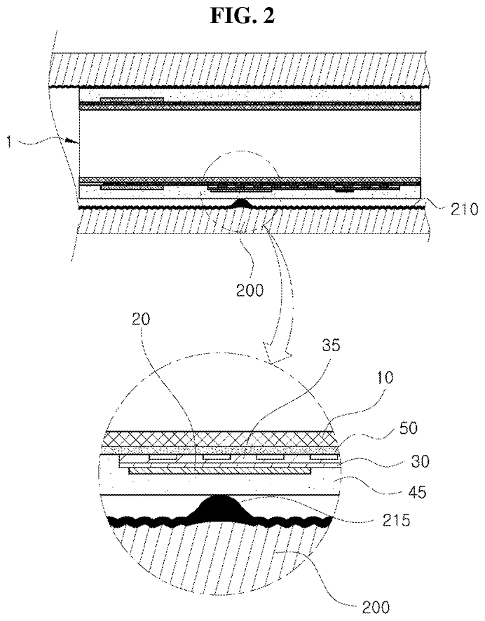

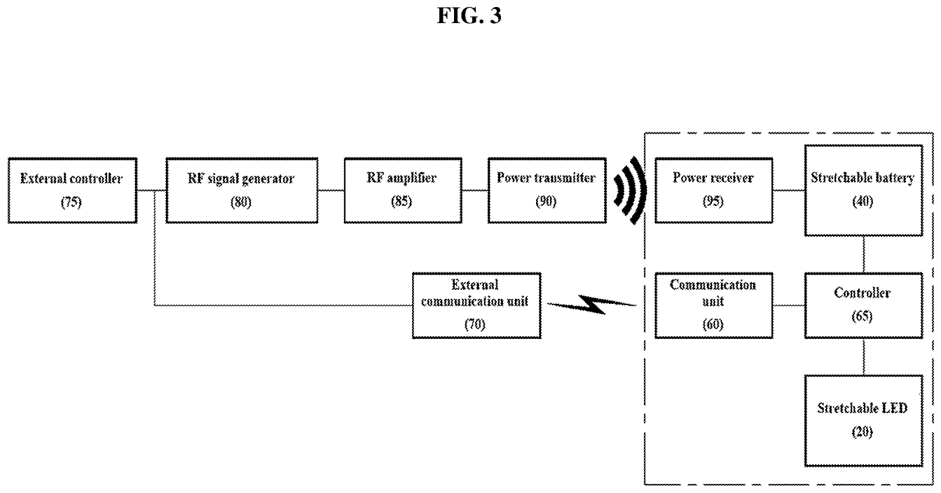

FIGS. 1 to 3 illustrate a medical self-expandable stent, according to an embodiment of the inventive concept.

As illustrated in the drawings, according to an embodiment of the inventive concept, a medical self-expandable stent 1 includes a stent body 10, a stretchable light emitting diode (LED) 20, a stretchable battery 40, and a thin-film formation part 45.

The stent body 10 has the shape of a cylindrical tube which is contractible and expandable. The wall surface of the stent body 10 has the shape of a mesh formed by crossing and weaving a plurality of spiral filaments.

The stent body 10 is contracted in the process of being inserted into a living tissue of a human body, such that an outer diameter of the stent body 10 is decreased. The stent body 10 is expanded in an operating position when a temperature is increased, such that the outer diameter of the stent body is increased. Accordingly, the stent body 10 inserted into the living tissue prevents the stenosis of the living tissue. In other words, the stent body 10 is self-expanded to emit heat when heat is applied from the stretchable LED 20.

The stent body 10 may be formed of a shape-memorial alloy that is self-expanded by the heat. In other words, the stent body 10 further includes magnesium (Mg) or is additionally coated with silicone including magnesium particles, thereby amplifying and emitting heat transferred from the stretchable LED 20. In addition, the stent body 10 may include a biodegradable material, such as magnesium (Mg) or polymers decomposed in a human body, which are naturally decomposed in the human body after a specific period of time is elapsed.

The stretchable LED 20 is stacked on the outer wall of the stent body 10 such that the stretchable LED 20 is spread while longitudinally extending.

The stretchable LED 20 may emit near infrared-ray and a specific wavelength and may perform a function of reducing glucose from a cell at a target point.

The stretchable LED 20 may regenerate a duodenal mucosal cell by using heat generated as a portion of optical energy is converted into thermal energy.

The stretchable LED 20 has a specific length, and may be disposed in a longitudinal direction of the stent body 10.

Although a plurality of stretchable LEDs 20 are illustrated according to the present embodiment, one or more stretchable LEDs 20 may be provided.

Meanwhile, the plurality of stretchable LEDs 20 may emit light having two or more wavelengths.

For example, the plurality of stretchable LEDs 20 may include a first stretchable LED 20a and a second stretchable LED 20b to emit light having wavelengths of 830 nm and 630 nm, respectively, which are near infrared rays serving as light for low level layer therapy (LLLT), and the two wavelengths exert an influence on adhesion molecule, thereby treating hyperglycemia. The first stretchable LED 20a for the wavelength of 830 nm and the second stretchable LED 20b for the wavelength of 630 nm may include operating modules independently operating. In addition, as illustrated in FIG. 1, the first stretchable LED 20a and the second stretchable LED 20b may be alternatively arranged in a circumferential direction of the stent body 10 while being spaced apart from each other.

Accordingly, the first stretchable LED 20a for the infrared ray having the wavelength of 830 nm is operated, and heat is emitted from the first stretchable LED 20a for the infrared ray having the wavelength of 830 nm, thereby removing a duodenal mucosa. The first stretchable LED 20a for the infrared ray having the wavelength of 830 nm has two functions of easily increasing the temperature of the heat emitted from the stent body 10, and of transferring heat to the duodenal mucosa such that the duodenal mucosa is directly ablated as heat is emitted from the infrared ray. The temperature, which is irradiated to a target part of the duodenal mucosa by the first stretchable LED 20a for the infrared ray having the wavelength of 830 nm, may be in the range of 44° C. to 90° C., and the irradiation time may be in the range of 10 minutes to one hour. In this case, the temperature of the first stretchable LED 20a is prevented from being excessively increased by a component of a radiating plate 35 to be described.

Thereafter, the operation of the first stretchable LED 20a for the infrared ray having the wavelength of 830 nm is stopped and a second stretchable LED 20b for the infrared ray having the wavelength of 630 nm is operated, such that the infrared ray is transmitted through the duodenal mucosa ablated by the heat and applied to a material, which increases blood glucose, thereby reducing the blood glucose. In addition, the second stretchable LED 20b for the infrared ray having the wavelength of 630 nm promotes the regeneration of the duodenal mucosa damaged by the heat emitted from the first stretchable LED 20a for the infrared ray having the wavelength of 830 nm. In this case, the second stretchable LED 20b prevents the temperature from being excessively increased by the radiating plate 35 to be described.

Meanwhile, such stretchable LED 20 may include a biodegradable material, such as a luminescent protein or a polymer having a pore, which is naturally decomposed in a human body after a specific period of time is elapsed.

In addition, the stretchable LED 20 may be controlled to be in the range of 44° C. to 90° C. in response to a control command of a controller 65 to be described. Accordingly, a cell related to diabetes and obesity may be destroyed at the target part of the living tissue, and an immune cell may be activated at the target part through a thermal action.

Meanwhile, the stretchable LED 20 may be disposed on a circuit board 30 stacked on the outer wall of the stent body 10. The circuit board 30 may include a circuit to drive the stretchable LED 20. The circuit board 30 may be formed of a material to be stretched by heat to correspond to that the stretchable LED 20 is longitudinally stretched.

In addition, the radiating plate 35 may be provided at a lower portion of the circuit board 30 to radiate heat transferred from the stretchable LED 20 to the circuit board 30.

As described above, as the radiating plate 35 is provided at the lower portion of the circuit board 30, the temperatures of the stretchable LED 20 and the circuit board 30 may be prevented from being increased to a specific temperature or more, such that the stretchable LED 20 and the circuit board 30 may be prevented from being damaged. In particular, as the stretchable LED 20 emits heat, the temperature at the target part is uniformly maintained. For example, the temperature at the target part may be maintained to be in the range of 44° C. to 90° C. In addition, as the temperature of the heat emitted from the stretchable LED 20 at the target part of the living tissue is maintained in the range of 44° C. to 90° C., thermal ablation may be performed.

In this case, although it is illustrated that the radiating plate 35 is stacked on the lower portion of the circuit board 30 according to the present embodiment, the inventive concept is not limited thereto. For example, the radiating plate 35 may be provided to be contactable with one area of the circuit board 30.

The stretchable battery 40 may be stacked to be longitudinally stretched on the outer wall of the stent body 10. For example, the stretchable battery 40 is interposed between the stent body 10 and the stretchable LED 20. In this case, the stretchable battery 40 may be stacked to be longitudinally stretched on the outer wall of the stent body 10 while forming the same plane with the stretchable LED 20.

The stretchable battery 40 is electrically connected to the stretchable LED 20 to supply electric energy to the stretchable LED 20.

The stretchable battery 40 may include a biodegradable material, such as paper and a polymer, which is naturally decomposed in the body of a patient after a specific period of time is elapsed. In particular, pores are formed in the stretchable battery 40 such that the stretchable battery 40 has a stretchable function.

The stretchable battery 40 may include a thin-film pouch cell or a flexible battery.

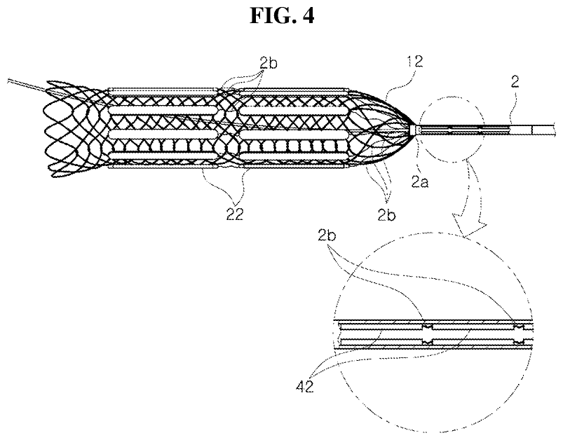

FIG. 4 is a perspective view illustrating a medical self-expandable stent, according to another embodiment of the inventive concept.

Referring to FIG. 4, a stretchable battery 42 is stacked to be longitudinally stretched on an outer wall of a stent body 12, and interposed between the stent body 12 and a stretchable LED 22. In this case, the stretchable battery 42 may be stacked to be longitudinally stretched on the outer wall of the stent body 12 while forming the same plane with the stretchable LED 22.

The stretchable battery 42 may be disposed in a stent inserting mechanism 2, and the length of the stent inserting mechanism 2 may generally have the length in the range of 180 cm to 230 cm. The stretchable battery 42 may be attached to the stent inserting mechanism 2, the stretchable LED 22 may irradiate light in the state that the stent body 12 having the stretchable LED 22 attached thereto is not completely spread, and then the stent body 12 and the stent inserting mechanism 2 may be removed.

As illustrated in the drawings, when the stent body 12 is spread only at a front portion thereof except for a rear portion thereof, a larger quantity of power may be supplied, as the stretchable battery 42 is disposed in a typical stent inserting mechanism (an inner tube or an outer sheath) having the diameter of 0.2 cm to 1 cm and the length of 180 cm to 230 cm, instead of the stent body 12. Accordingly, the stretchable LED 22 of the stent body 12 may more efficiently irradiate light.

In addition, when the stretchable LED 22 or the stretchable battery 42 is attached to the stent body 12, the thickness of the stent body 12 may be increased. Accordingly, as the stretchable battery 42 is disposed in the stent inserting mechanism 2, the outer diameters of the stretchable LED 22 and the stent body 12 may be reduced.

In this case, the stent body 12 may be connected to an open end portion of the stent inserting mechanism 2 through a joint part 2a.

The stretchable battery 42 attached to the stent inserting mechanism 2 connects the stretchable LED 22 attached to the stent body 12 to a power supply line 2b to supply power (wireless control through Bluetooth). The stent body 12 may be implemented in the form of a catheter for light irradiation of the stretchable LED 20 using the stent body 12, in which the stent body 12 is partially expanded without being inserted into the body and placed at the stent inserting mechanism 2 during the procedure as in the drawing and then both the stent body 12 and the stent inserting mechanism 2 are removed after the procedure.

The stretchable battery 40 may have a cylindrical shape or a rectangular shape. A plurality of stretchable batteries 40 may be provided on the outer wall of the stent body 10 to correspond to the number of stretchable LEDs 20, or a single stretchable battery 42 may be provided to expand the surficial area of the stretchable battery 42.

In addition, the stretchable battery 40 may have the shape in which a plurality of pores are formed through the stretchable battery 40, such that heat emitted from the stretchable LED 20 is easily transferred to the stent body 10.

The thin-film formation part 45 includes a thin film formed to fix the stretchable LED 20, the circuit board 30, the radiating plate 35 and the stretchable battery 40 to the stent body 10.

The thin-film formation part 45 may include transparent silicone that is self-expanded by heat, transmits light, and transmits heat. In addition, the thin-film formation part 45 may include a biodegradable polymer naturally decomposed in the body after a specific period of time.

The thin-film formation part 45 includes a thin film formed while surrounding the stretchable LED 20, the circuit board 30, the radiating plate 35, and the stretchable battery 40, through electrospraying or electrospining, thereby fixing the stretchable LED 20, the circuit board 30, the radiating plate 35, and the stretchable battery 40 to the outer wall of the stent body 10.

The thin-film formation part 45 may not only protect the stretchable LED 20, the circuit board 30, the radiating plate 35, and the stretchable battery 40 from electrical short circuit or corrosion, but prevent internal substances of the stretchable LED 20, the circuit board 30, the radiating plate 35, and the stretchable battery 40 from leaking into the living tissue.

In addition, the thin-film formation part 45 may further include magnesium. For example, magnesium particles, preferably, magnesium particles having the size of 250 nm or more, are incorporated into a silicone constituting the thin-film formation part 45 and electrospraying or electrospining is performed, thereby forming the thin film. Accordingly, the heat emitted from the radiating plate 35 is transferred to the magnesium particles to maximize the hyperthermia therapy for the target part of the living tissue.

In this case, the stretchable LED 20 may be provided to be exposed from the thin-film formation part 45 to maximize the irradiation of light at the target part of the living tissue.

In addition, according to an embodiment of the inventive concept, the medical self-expandable stent 1 may further include an auxiliary thin-film formation part 50.

The auxiliary thin-film formation part 50 may be self-expanded by heat and may include a transparent silicone to transmit light and transfer heat. In addition, the auxiliary thin-film formation part 50 may include a biodegradable polymer naturally decomposed in the body after a specific period of time is elapsed.

The auxiliary thin-film formation part 50 includes a thin film formed between the outer wall of the stent body 12, and the stretchable LED 20, the circuit board 30, the radiating plate 35, and the stretchable battery 40 to fix the stretchable LED 20, the circuit board 30, the radiating plate 35, and the stretchable battery 40 to the stent body 10.

The auxiliary thin-film formation part 50 may prevent internal substances of the stretchable LED 20, the circuit board 30, the radiating plate 35, and the stretchable battery 40 from leaking out of the stent body 10 and being introduced into the living tissue.

In addition, the auxiliary thin-film formation part 50 may further include magnesium (Mg). For example, magnesium particles, preferably, magnesium particles having the size of 250 nm or more, are incorporated into a silicone constituting the auxiliary thin-film formation part 50, thereby forming the thin film through electrospraying or electrospining. Accordingly, the heat emitted from the radiating plate 35 is transferred to the magnesium particles to maximize the hyperthermia therapy for the target part of the living tissue.

In addition, according to an embodiment of the inventive concept, the medical self-expandable stent 1 may further include a communication unit 60 and the controller 65.

The communication unit 60 and the controller 65 are stacked on the stent body 10, for example, the circuit board 30.

The communication unit 60 may receive a control command from the outside of a human being through wireless communication. The communication unit 60 makes communication with an external communication unit 70 provided at the outside, and the external communication unit 70 makes communication with the communication unit 60 in response to the control command of an external controller 75 provided at the outside. In this case, the communication unit 60 may be provided in various shapes such as multiple-input multiple-output (MIMO) antennas.

The controller 65 is electrically connected to the stretchable battery 40 and the communication unit 60. The controller 65 controls the stretchable battery 40 in response to a control command received from the communication unit 60 to turn on or off the stretchable LED 20. Accordingly, an operator may conveniently operate the stretchable LED 20, at the outside.

In addition, according to an embodiment of the inventive concept, the medical self-expandable stent 1 may further include a radio frequency (RF) signal generator 80, an RF amplifier 85, a power transmitter 90, and a power receiver 95 such that power is wirelessly supplied to the stretchable battery 40 at the outside of the body.

The RF signal generator 80, the RF amplifier 85, and the power transmitter 90 are provided outside without being inserted into the body together with the stent body 10, and are controlled in response to a control command of the external controller 75 provided outside. In addition, the power receiver 95 is electrically connected to the stretchable battery 40 and provided on the circuit board 30 stacked on the stent body 10.

The RF signal generator 80 generates an RF signal.

The RF amplifier 85 amplifies an RF signal output from the RF signal generator 80 to specific power.

The power transmitter 90 wirelessly transmits the RF signal amplified to the specific power in the RF amplifier 85.

The power receiver 95 converts the RF signal transmitted from the power transmitter 90 into electric power to be supplied to the stretchable battery 40. The power receiver 95 may include a plurality of induction coils connected in series or in parallel.

As described above, the medical self-expandable stent 1 may further include the RF signal generator 80, the RF amplifier 85, the power transmitter 90, and the power receiver 95 to wirelessly transmit power to the medical self-expandable stent 1 inserted into the human body, such that the target part of the living tissue may be consecutively treated.

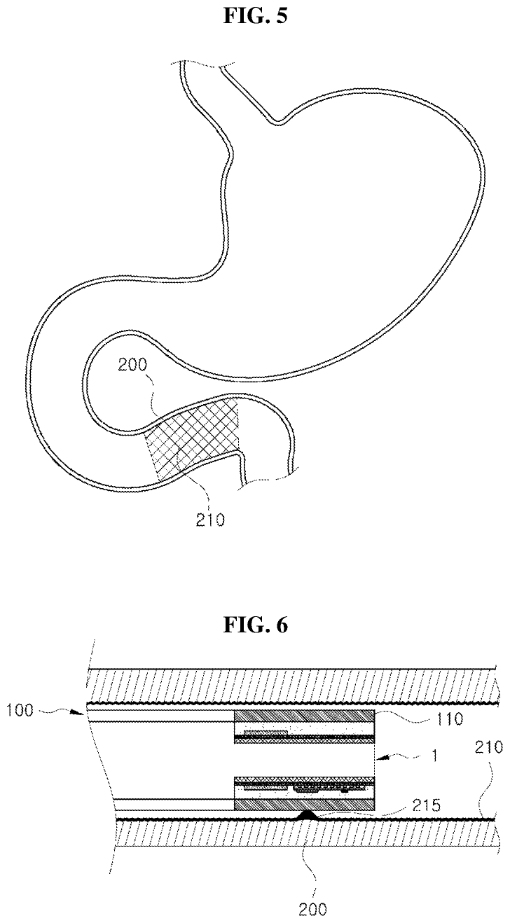

The following description will be made, with reference to FIGS. 5 to 10, regarding that a treatment procedure is performed by inserting the medical self-expandable stent 1 according to an embodiment of the inventive concept into part 2 of a duodenum 200, which serves as a target part 215 of a living tissue.

First, as illustrated in FIG. 6, the medical self-expandable stent 1 is positioned to be overlapped with the target part 215 by inserting and moving a head 110 of an endoscope 100, which is equipped with the medical self-expandable stent according to an embodiment of the inventive concept, into the target part 215 of the duodenum 200.

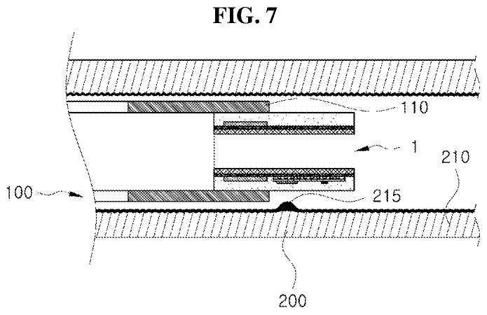

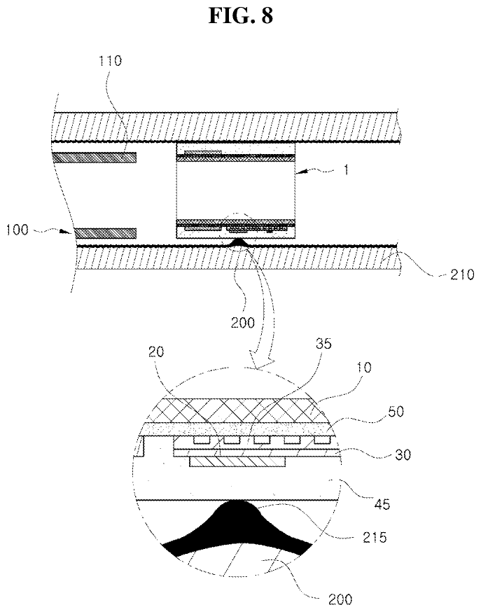

Next, as illustrated in FIG. 7, the endoscope 100 is withdrawn out of the duodenum 200 in the state that the medical self-expandable stent 1 is overlapped with the target part 215, thereby seating the medical self-expandable stent 1 onto the target part 215 as illustrated in FIG. 8.

In this case, when the medical self-expandable stent 1 is seated on the target part 215, the medical self-expandable stent 1 expands in the radial direction from the medical self-expandable stent 1 such that the outer diameter of the medical self-expandable stent 1 is increased, thereby preventing the stenosis at the target part 215 of the duodenum 200.

In addition, when the control command is transmitted to the controller 65 from the outside of the body through wireless communication such that the stretchable LED 20 emits light, the controller 65 supplies the power of the stretchable battery 40 to the stretchable LED 20 and turns on the stretchable LED 20.

As a portion of the optical energy supplied to the stretchable LED 20 is converted into thermal energy, the stretchable LED 20 emits heat.

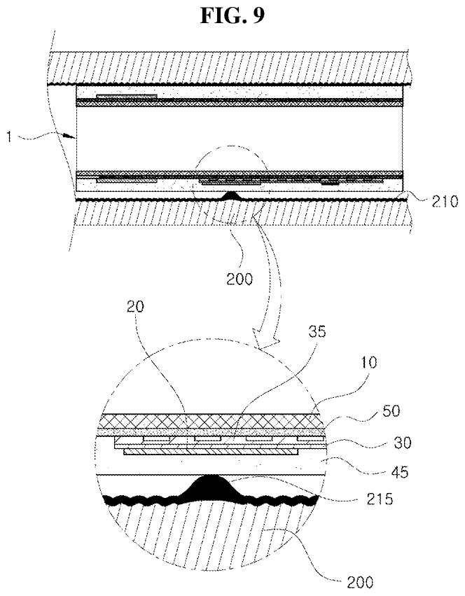

As the stretchable LED 20 emits heat, the stretchable LED 20, the circuit board 30, the radiating plate 35, the stretchable battery 40, the thin-film formation part 45, the auxiliary thin-film formation part 50, and the stent body 10 are self-stretched or expanded in the longitudinal direction of the stent body 10 by the heat of the stretchable LED 20, and then uniformly transfer heat to the target part as illustrated in FIG. 9.

In this case, some of the heat emitted from the stretchable LED 20 is transferred to the stent body 10 through the circuit board 30 and the radiating plate 35, and the stretchable LED 20 maintains a specific temperature.

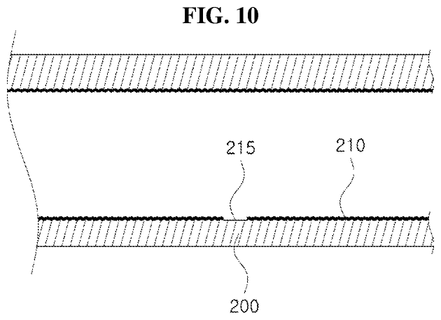

As the stretchable LED 20 irradiates light while transferring heat toward the target part 215 for specific time, the target part 215 of the duodenum 200 is ablated as illustrated in FIG. 10.

In particular, an adhesion molecule is influenced by the wavelength emitted from the stretchable LED 20 to product the effect of hyperglycemia treatment. In addition, the tumor at the target part 215 of the duodenum 200 may be reduced by the heat from the stretchable LED 20, and the immune cells at the target part 215 may be activated.

When light is irradiated and heat is applied toward the target part 215 for a specific period of time, the medical self-expandable stent 1 seated at the target part 215 is withdrawn out of the body using the endoscope 100 and recovered.

Accordingly, the target part 215 of the duodenum 200 is ablated by using the optical energy of the stretchable LED 20 to treat a mucous membrane 210 of the duodenum 200 by regenerating the mucous membrane 210 without the damage to the tissue of the mucous membrane 210 of the duodenum 200 and to prevent the stenosis of the duodenum 200, such that treatment complications may be reduced.

Meanwhile, although the above-described embodiment has been described in that the medical self-expandable stent 1 is seated at the target part 215 of the duodenum 200 or recovered from the target part 215 by using the endoscope 100, the inventive concept is not limited thereto. The medical self-expandable stent 1 is mounted at the stent inserting mechanism (not illustrated) in the state that the medical self-expandable stent 1 is compressed, such that the medical self-expandable stent 1 is seated at the target part 215 of the duodenum 200 or recovered from the target part 215 by using the stent inserting mechanism.

In addition, according to an embodiment of the inventive concept, when the target part 215 of the duodenum 200 is treated using the medical self-expandable stent 1, the RF signal is generated from the RF signal generator 80 in response to the control command of the external controller 75, and amplified to specific power by the RF amplifier 85, and then the amplified RF signal is wirelessly transmitted to the power receiver 95 through the power transmitter 90. The power receiver 95 may convert the RF signal transmitted from the power transmitter 90 into power to be charged into the stretchable battery 40, thereby consecutively treating the target part 215 of the duodenum 200.

As described above, according to the inventive concept, the medical self-expandable stent is inserted into the target part of the duodenum, and the optical energy generated from the stretchable LED provided in the medical self-expandable stent is irradiated to the target part of the duodenum for a specific time, such that the duodenal mucosa is regenerated through the optical energy and the thermal energy, thereby reducing diabetes-related substances. The procedure may be conveniently performed using the endoscope, and the stent body prevents the duodenal stenosis, thereby reducing treatment complications related to the duodenal mucosal regeneration.

In addition, according to the inventive concept, the medical self-expandable stent is formed of a biodegradable material, such that the medical self-expandable stent inserted into the body is naturally decomposed after a specific period of time is elapsed. Accordingly, the medical self-expandable stent does not need to be recovered through an endoscopic mechanism or a stent inserting mechanism.

According to the inventive concept, the treatment may be performed by ablating the mucosal cell at the target part of the living tissue using optical energy of the stretchable LED to regenerate the mucosal cell of the duodenum without the damage to the duodenum tissue, and the complications resulting from the treatment may be reduced by preventing the duodenal stenosis.

In addition, the target part of the living tissue may be consecutively treated by transmitting the power to the stent inserted into the human body wirelessly or through the battery.

The effects of the inventive concept are not limited to the above, but other effects, which are not mentioned, will be apparently understood to those skilled in the art.

Although embodiments of the inventive concept have been described with reference to accompanying drawings, those skilled in the art should understand that various modifications are possible without departing from the technical scope of the inventive concept or without changing the technical sprite or the subject matter of the inventive concept. Therefore, those skilled in the art should understand that the technical embodiments are provided for the illustrative purpose in all aspects and the inventive concept is not limited thereto.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A medical self-expandable stent comprising:
a stent body self-stretched or expanded by heat and configured to emit the heat;
a stretchable light emitting diode (LED) stacked to be longitudinally spread on the stent body and configured to emit the heat by converting electric energy into optical energy; and
a thin-film formation part including a thin film to fix the stretchable LED to the stent body, and self-expanded by the heat.

2. The medical self-expandable stent of claim 1, further comprising:
a stretchable battery provided to be surrounded by the thin-film formation part and longitudinally spread on the stent body, and configured to supply the electric energy to the stretchable LED.

3. The medical self-expandable stent of claim 1, further comprising:
a communication unit configured to be stacked on the stent body and receive a control command from an outside through wireless communication; and
a controller configured to be stacked on the stent body and turn on or turn off the stretchable LED in response to the control command received from the communication unit.

4. The medical self-expandable stent of claim 1, further comprising:
an auxiliary thin-film formation part including a thin film between the stretchable LED and the stent body, and self-expanded by the heat.

5. The medical self-expandable stent of claim 2, further comprising:
a radio frequency (RF) signal generator configured to generate an RF signal;
an RF amplifier configured to amplify the RF signal, which is output from the RF signal generator, to specific power;
a power transmitter configured to wirelessly transmit the RF signal amplified to the specific power; and
a power receiver configured to convert the RF signal received from the power transmitter into power and to supply the power to the stretchable battery.

6. The medical self-expandable stent of claim 1, further comprising:
a circuit board on which the stretchable LED is mounted; and
a radiating plate provided on the circuit board, and configured to radiate the heat transferred from the stretchable LED to the circuit board.

7. The medical self-expandable stent of claim 1, wherein a plurality of stretchable LEDs are provided, and emit light having mutually different wavelengths.

8. The medical self-expandable stent of claim 2, wherein the stretchable battery includes a thin-film pouch cell or a flexible battery.

9. The medical self-expandable stent of claim 1, wherein the thin-film formation part is formed by performing electrospraying or electrospining for silicone.

10. The medical self-expandable stent of claim 9, wherein the thin-film formation part further includes magnesium (Mg).

11. The medical self-expandable stent of claim 4, wherein the auxiliary thin-film formation part is formed by performing electrospraying or electrospining for silicone.

12. The medical self-expandable stent of claim 11, wherein the auxiliary thin-film formation part further includes magnesium (Mg).
